(19) United States
(12) Patent Application Publication
(10) Pub. No.: US 2002/0104531 A1
(43) Pub. Date: Aug. 8, 2002

(54) Pediatric inhalation device

(76) Inventor: Rand Malone, Thomasville, GA (US)

Correspondence Address: Brian D. Bellamy, P.O. Box 1997, Thomasville, GA, 31799-1997, US

(21) Appl. No.: 10/051,208

(22) Filed: Jan. 18, 2002

Related U.S. Application Data
(60) Provisional application No. 60/262,329, filed on Jan. 18, 2001.

(51) Int. Cl. A61M 11/00; A62B 7/00; A62B 18/02

(52) U.S. Cl. 128/200.23; 128/203.12; 128/205.25; 128/206.21

(57) ABSTRACT

A pediatric inhalation device and method including a auxiliary device having an inlet, an outlet, and a chamber. An administration device attached to the outlet, and an inflatable mask of soft pliable material attached to the administration device. The administration device comprises a T-adaptor with an inhalation valve and exhalation valve of about equal size that are visible to the adult observer. A whistle provided on the pediatric inhalation device is capable of making an audible sound during desired inhalation by the child.

### DOMESTIC PRIORITY CLAIM

The priority of U.S. Provisional Application No. 60/262,329, filed Jan. 18, 2001 is claimed.

### BACKGROUND OF THE INVENTION

The present invention relates to an inhalation device for inhalation of a pharmaceutically active substance from a reservoir. In particular, the invention relates to a pediatric inhaler combination that enables young children to inhale medicaments with a lower rate of failure.

Asthma is a rapidly developing health problem in the United States with infants and toddlers comprising the majority of newly diagnosed patients. Therapy for asthma is now based on healing and then preventing lower airway inflammation with anti-inflammatory therapy delivered directly to the lungs. The most viable way to accomplish this is with corticosteroid metered dose inhalers (MDIs) delivered through space chambers that hold the aerosol cloud until the young child is able to inhale it.

Very young children are unable to hold mouth pieces securely between their lips so face masks have been attached to the chambers that will then fit around the mouth and/or nose of the child. One-way valves were developed to place between the chamber and the mask to prevent the emitted aerosol from striking the face before the child was inhaling. Over time it became apparent that children were unable to exhale after inhalation without breaking the seal of the mask on their face. If the parent was holding the mask tightly in place, the child had to wiggle his head violently in order to breathe out. A face mask was developed that had a small exhalation valve built in that was a definite improvement.

Therapy for asthma in young children remains difficult because young children often end up in a power struggle with their parents due to inadequacies in the MDIs currently marketed. Parents rightfully become frustrated, and, as a result, poor compliance with medications, especially those that are inhaled, is the number one reason for sub-optimal outcomes in asthma management.

The face masks that are currently available on the market for use with MDIs are inadequate for use with young children for a variety of reasons. First, the face masks do not conform well to the face of the small child. The material of the masks currently used with MDIs is not very pliable and cannot be adjusted to fit the various shapes and sizes of children's faces. Thus, in order to form a good seal, the mask must be pressed very firmly onto the child's face. However, pressing the mask to a child's face is uncomfortable and causes a smothering sensation that children resist. This fighting against the mask makes the parent press even more firmly causing even more pressure against the face. Often one parent must pin the arms of the child down while another adult holds the child's head still and applies the mask. This makes the child cry. Studies have now shown that crying children have very rapid inspiratory rates that cause the aerosol particles to be delivered in the pharynx, throat and proximal bronchial tree instead of the lower airways where asthma occurs and medication is needed.

The inadequacy of inhalation device face masks for children is furthered by the rapid growth of children that necessitates various sizes of masks be used until the child is old enough to use a mouth piece. One face mask does not work throughout the first three to four years of life.

A further inadequacy of current MDIs for use with children is that the exhalation valves on the masks are much smaller than the inhalation valves. In asthmatic children air flow obstruction occurs on exhalation much more than on inhalation. Expiratory times are markedly increased in asthma because resistance to exhalation is increased by swelling of the airway and excess mucous production. Chronic air trapping in the lungs also decreases pulmonary elastic recoil which further decreases expiratory air flow. All of the pediatric inhalation products on the market have exhalation valves that are smaller than those for inhalation which increases resistance with exhaling. This is exactly opposite of what an asthmatic would benefit from and increases the smothering sensation associated with not being able to adequately dispel air from the device.

Yet a further inadequacy of current MDIs for use with infants and young children exists. In particular, it is generally difficult to discern whether or not any inhaled medication is delivered to the toddler or infant with devices currently on the market. On most of the devices the inhalation valve is hidden inside the spacer or face mask. Some of the valves can be viewed at an oblique angle through the walls of the canister. However, these valves open away from the observer and movement can be difficult to discern. Further, exhalation valves are small making it difficult for patients to see their movements at all.

### SUMMARY OF THE INVENTION

The device of the present invention overcomes the inherent problems in administering inhaled medicaments to young children and infants. The invention resolves these problems and improves delivery of MDI aerosols to infants, toddlers and other children too young to properly use a mouth piece or common face mask. The invention improves the design of the now common aerosol chamber with mask and one-way valves used to deliver metered dose aerosols to infants and toddlers with asthma.

The most significant drawback to spacer devices currently available is that they are not designed in such a way as to promote cooperation from the child in taking their own medication. A dramatic improvement would employ a low flow inspiratory horn that honks loudly when the child inspires. All of those horns (also referred to as whistles) currently available honk when the flow rate is too high. The inventor has found that young children love to honk horns, and this can be used as an incentive to teach them to take the medication without assistance. Simply honking the device and throwing it on the floor can often achieve this end. Children are so inquisitive, that if left to themselves, they will usually try to honk the spacer on their own.

The inventor has found that employing an inflatable mask that is very soft and pliable allows the mask to easily seal with minimal force applied by the child when they are attempting to reproduce the sound themselves. Further, it has been found that provide a right angle between the mask and the chamber makes it easy for the child to hold the device for self-delivery. In an office setting it is best if children can be taught to administer medication to themselves, as this makes delivery at home much more practical. Ideally, before the parent leaves the office, the child will already be using the device without assistance, which dramatically improves compliance in the future.

Accordingly, an auxiliary device comprising a holding chamber (such as the Ace brand chamber) having an inlet adapted to receive an MDI device is provided. The chamber has a small volume sufficient to hold the aerosol cloud, but that limits dead space within the chamber. The holding chamber comprises clear plexiglass for viewing within the chamber.

An outlet is provided in the auxiliary device chamber for attaching an administration device for communicating with the nose and/or mouth of an infant or young child. The device provides a coaching whistle on the back of the device which will make a honking sound when air is pulled through at a particular flow rate. The flow rate required to make the whistle sound has been reduced so that young infants down to six months of age can usually cause the whistle to sound.

The administration device is attached to the outlet and is made of transparent material. The administration device includes a T-adaptor (such as the Nif-Tee brand adaptor) having a first tubular portion with a first inhalation valve at the first end thereof and a second exhalation valve at the opposing second end thereof. The inhalation and exhalation valves are of about equal size and are very thin and pliable allowing them to open at very low inhalation and exhalation flow rates. A second tubular portion forming an open base extends perpendicular to the center of the first tubular portion to form the t-shaped adaptor. A mask adapted for placement over a young child's nose and mouth is provided to attach to the open base of the T-adaptor.

In this invention, a soft plastic inflatable mask (such as the model known as the Vent Mask VR-3 100-40) is used, which has very thin walls that contact the patient's face. When the mask is inflated to about half full, the walls of the face mask are pliable and will fit the contour of any size or shape of face without any leaking of air. One size of the mask will fit all infants and young children.

The present invention provides the first chamber and mask device for delivering MDI aerosols that is designed to be successfully self administered by children under the age of three to four years of age.

### BRIEF DESCRIPTION OF THE DRAWINGS

FIG. 1 is a perspective view of the pediatric inhalation device.

FIG. 2 is an exploded view of the pediatric inhalation device.

FIG. 3 is an exploded perspective view of the diaphragm of FIG. 2.

FIG. 4 is a perspective view of the horn of FIG. 2.

FIG. 5 is a side elevational view of the pediatric inhalation device applied to a child's face and showing the flow of the medicament.

### DETAILED DESCRIPTION OF THE DRAWINGS

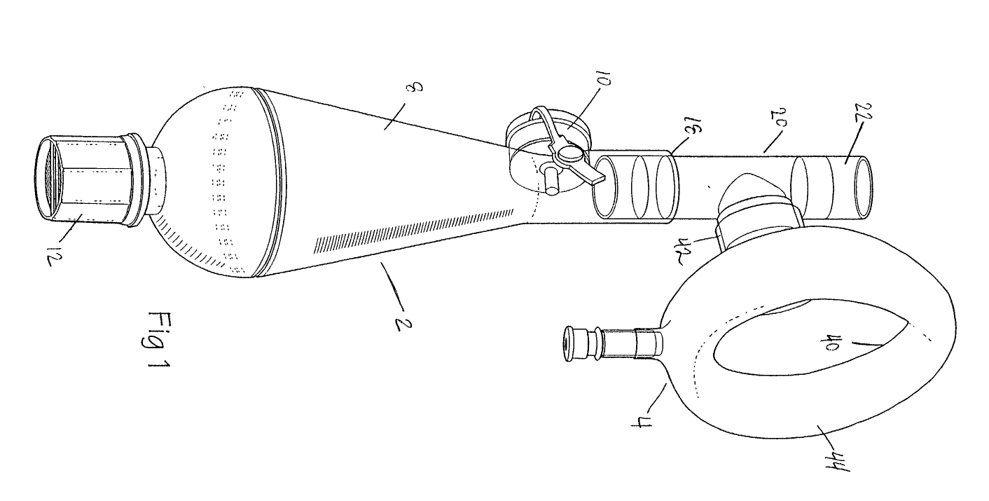

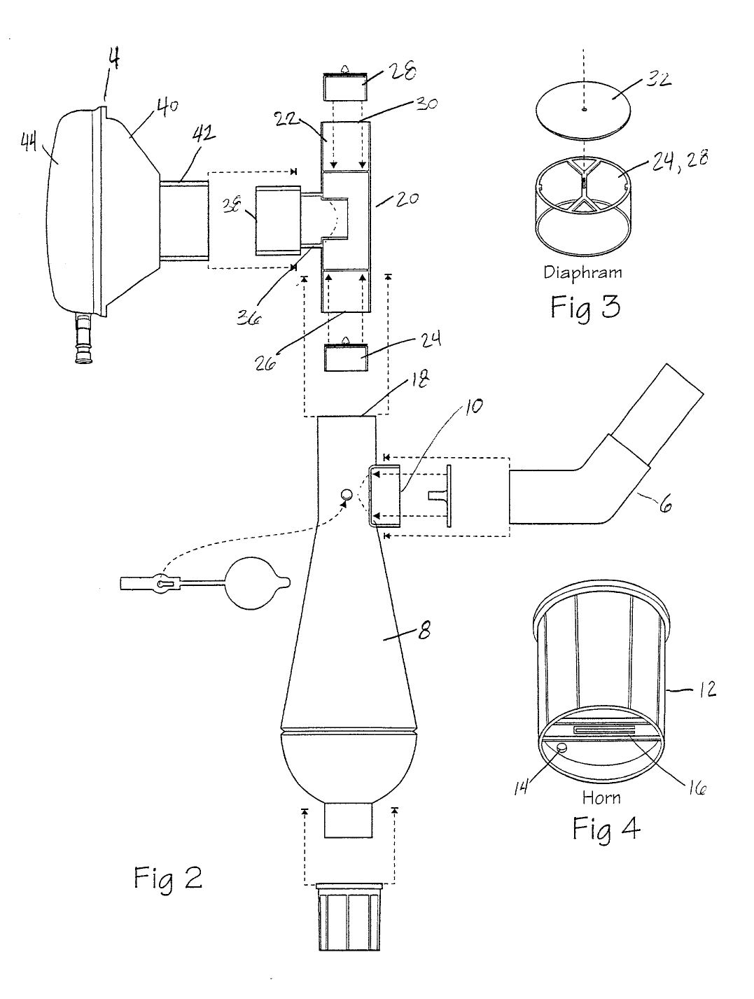

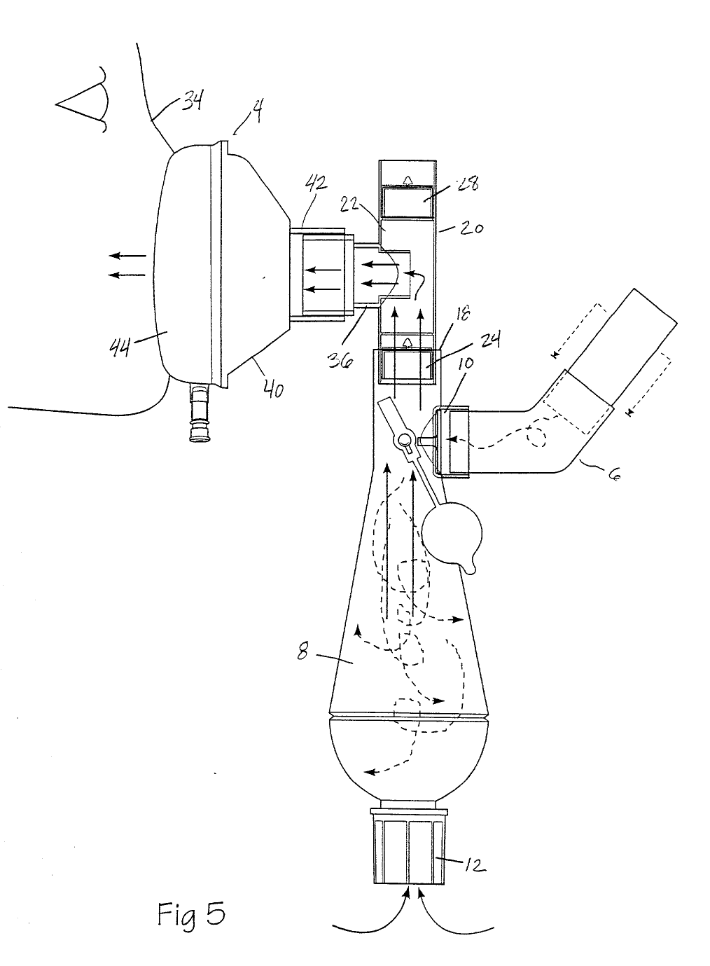

Referring now to the drawings, FIG. 1 illustrates an embodiment of the present invention. The invention includes a combination of an auxiliary device 2 and an administration device 4 adapted for use with metered dose inhalers (MDIs) for delivering corticosteroids or the like to infants and young children under the age of three or four years, referred to generally herein as children.

The auxiliary device 2 has an inlet 10 adapted to receive an MDI device 6. Upon actuation of the MDI 6, a dose of a medicament is delivered through the inlet into a holding chamber 8. The holding chamber 8 is made of transparent plexiglass or other suitable transparent material for viewing within the chamber, and the holding chamber 8 is constructed having a volume sufficient to hold the aerosol cloud. Excess volume is eliminated in the design of the holding chamber 8 to limit the amount of dead space in the chamber, which optimizes the delivery of an aerosol to children with very low tidal volumes.

In the present invention, the Ace™ chamber is used for the holding chamber 8 in a preferred embodiment of the invention because the Ace™ chamber is designed for minimal dead space when intervened in respiratory tubing of intubated patients. Other advantages of the Ace™ chamber include that the MDI 6 can be fired directly into the chamber 8 without using the plastic actuator that accompanies the MDI 6, and the Ace™ chamber provides an inlet 10 that makes all MDIs attach equally well. The plastic mouth pieces or orifices on MDIs 6 are either oval or round depending on the brand. Most chambers are designed for connection to either the oval orifice or the round orifice, but not both. The Ace™ chamber provides the convenience of attaching to all MDI orifices regardless of mouth piece shape because it attaches directly to the cannister stem.

As illustrated in the drawings a coaching whistle 12 is provided on the bottom end of the chamber 8. The coaching whistle 12, also referred to as a horn, makes a honking sound when air is pulled through the inhalation device 2 at a particular flow rate. In the present invention, the inventor has found distinguishing advantages in reducing the flow rate required to make the whistle 12 sound. In particular, the flow rate is reduced in the illustrated device by occluding the auxiliary air flow port 14 so that all the inspired air must travel through the sound producing orifice 16. By reducing the flow rate required to cause the whistle sound, young infants down to six months of age can make the whistle 12 sound by inhaling normally. The whistle 12 gives young children an incentive to try the inhalation device and to learn how to use the device and inhale to receive the medicament.

The inventor has found the reduction of the flow rate to sound the whistle to have surprising value. In fact, prior art horns and whistles have been designed to wam a parent that a child's flow rate is too fast for good pulmonary deposition. Thus, children have previously been discouraged from initiating the whistle sound. However, with children less than three years of age breathing too fast is not critical to good pulmonary deposition of the medicament. Instead, the critical challenge is getting the child to use the device without struggling and crying in that such behavior dramatically reduces pulmonary deposition. Using the whistle 12 as an incentive and indicator of inhalation greatly enhances a young child's ability to learn and use the inhalation device. Further, the whistle 12 provides audible verification for a parent that the inhalation occurred, which gives the parent peace of mind.

The auxiliary device 2 includes an outlet 18 that is provided in the chamber 8 for attaching an administration device 4. The administration device 4 provides a means for the chamber 8 to communicate with the nose and/or mouth of an infant or young child.

As shown in the present embodiment, the administration device 4 includes a T-shaped adaptor 20 made of transparent material. Similar T-adaptors 20 have been designed to wye-in various types of tubing on ventilators for intubated patients, but have not been used with pediatric inhalation devices. The inventor has found previously undiscovered and significant advantages of using the T-adaptor 20 with the present pediatric inhalation device as described herein.

The T-adaptor 20 comprises a first tubular portion 22 that houses an inhalation valve 24 at a first end 26 thereof. The first end 26 of the T-adaptor attaches to the outlet 18. The first tubular portion 22 houses an exhalation valve 28 at the opposing second end 30 thereof. In the preferred embodiment, the inhalation valve 24 and exhalation valve 28 are of about equal size, whereas in prior art designs the exhalation valve is generally smaller than the inhalation valve. Further, the exhalation valve 28 is larger than exhalation valves in prior art auxiliary devices. The design of the exhalation valve 28 to be as large as the inhalation valve 24 allows young children who have expiratory obstruction from asthma to exhale more easily, preventing dyspnea and potential struggling or crying. Also, the preferred valves 24 and 28 include a diaphragm 32 that is very thin and pliable so that the diaphragm 32 opens at very low inhalation and exhalation flow rates. These features are especially beneficial in using the inhalation device with small infants having rapid respiratory rates who need minimal resistance to allow adequate exhalation.

Providing still further benefits of the invention and in particular the use of the T-adaptor 20, both valves 24 and 28 are encased within a transparent plastic material. The design and orientation of the T-adaptor 20 in the invention makes both valves 24 and 28 clearly visible from any angle and allows a parent to see the valves' 24 and 28 motions with respiration. By providing visual confirmation of respiration, the highly visible arrangement of T-adaptor 20 and valves 24 and 28 assures that the administration device 4 is applied properly to a child's face 34 and that medication is being delivered to the child. Further, an adult can easily count the valve movements so that desired number of respirations can occur to fully withdraw medication for each actuation of the MDI 6. This is particularly important in very small infants who routinely are unable to sound the inspiratory whistle.

The T-adaptor 20 includes a second tubular portion 36 having an aperture at the terminal end forming an open base 38 for the T-adaptor that extends perpendicular to the center of the first tubular portion 22, thus forming the T-shaped adaptor 20. The T-adaptor 20 can be removed from the administration device 4 and treated as a separate part for easy cleaning and replacement. It is especially advantageous that either the T-adaptor 20, the administration device 4, or the whistle 12 can be replaced without replacing an entire inhalation device because of the expensive cost of the complete devices.

The administration device 4 includes a mask 40 having an adapter 42 to attach the mask 40 to the open base 38 of the second tubular portion 36. The base 38 on the T-adaptor 20 is arranged such the base 38 is disposed at a right angle to the chamber 8, which makes the chamber easier to hold by a child.

Although, several advantages of the administration device 4 are provided herein that are available with the use of any suitable mask, the inventor has found additional benefit in using a inflatable mask 40 constructed of a soft pliable plastic bladder for the pediatric inhalation device taught by the invention. Such inflatable masks were previously designed and taught for use with bag/mask resuscitation of patients in cardiopulmonary arrest and also used in anesthesia. The advantages of the inflatable mask 40 for use with pediatric inhalers for MDIs 6 has not been recognized previously.

The inflatable mask 40 has an includes an inflatable bladder having very thin walls that are highly pliable. With the recommended inflation of the mask 40 to about half full, the pliable walls 44 of the mask 40 will fit the contour of any size or shape of face. Therefore, one size of the mask 40 will fit all infants and young children to age four without any leaking of air. More particularly, minimum pressure needs be applied by a child to seal the mask 40 against his or her face 34 because the walls 44 are so pliable, which allows a child to cause the whistle 12 to sound without excessive force against the face 34. Less application of force by an adult against a child's face 34 translates into less fear by the child and less struggle and crying with a more veritable attempt by the child to inhale the medicine from the chamber 8. In other words, the inflatable mask 40 leads to increased compliance by the child and improved lung deposition of the medicine. As parents leave the controlled environment of the doctor's office, the improved inhalation device with inflatable mask 40 allows self administration of the inhalation device by children with less error and will usually require only one parent to be present versus two with prior pediatric inhalation devices.

Another aspect of the present inhalation device is that the snug fit of the mask 40 when only partially filled with air dramatically decreases the dead air space with the mask itself when applied to the face 34. As discussed previously, the elimination of dead air space is significant in better delivery of medicine through the inhalation device. The reduction of dead space in the mask 40 is a significant advantage over many other masks on the market. In particular, small children have very low tidal volumes, and studies have recently shown that much more aerosol can be delivered to young children if the dead space with the chamber 8 is small. The concept of increased delivery of an aerosol to young children through reduction of dead space is equally applicable to reduction of dead space in the area between the mask 40 and face 34.

The present invention provides the first inhalation device having a chamber 8 and mask 40 for delivering MDI aerosols that is designed to be successfully self administered by children under the age of three to four years of age. Clinical trials have proven that more than 70% of the children over 17 months of age can hold the device to their face 34 and honk the coaching whistle 12 without a parent's help. Also, the vast majority of children two years and older can actuate the MDI device 6 without any parental input. Thus, the features of the present invention dramatically increases compliance with anti-inflammatory medications and allows superior asthma treatment outcomes over prior art devices.

I claim:

1. A pediatric inhalation device comprising:
a auxiliary device including an inlet, an outlet, and a chamber;
an administration device attached to the outlet; and
an inflatable mask attached to the administration device.

2. A pediatric inhalation device as in claim 1 including a coaching whistle that creates an audible sound during inhalation by a child between six months of age and four years old during effective administration of medicament from the pediatric inhalation device.

3. A pediatric inhalation device as in claim 1 in which the administration device includes a T-adaptor attached to the outlet of the auxiliary device, the T- adaptor having an inhalation valve, an exhalation valve, and a mask adaptor.

4. A pediatric inhalation device as in claim 3 in which the inhalation valve and the exhalation valve of the T-adaptor are of about equal size.

5. A pediatric inhalation device as in claim 3 in which the exhalation valve of the T-adaptor is operable by the flow of air produced by the normal exhalation of an infant.

6. A pediatric inhalation device as in claim 3 in which the inhalation and exhalation valves of the T-adaptor are encased in a transparent plastic material.

7. A pediatric inhalation device as in claim 3 in which the T-adaptor includes a first tubular portion having a first end that attaches to the outlet of the auxiliary device and the first end contains the inhalation valve therein, and the first tubular portion having a second end which contains the exhalation valve therein, and the T-adaptor further includes a second tubular portion attached perpendicularly to the first tubular portion and having an aperture at a terminal end thereof that forms a base of the mask adaptor for the connection of the inflatable mask.

8. A pediatric inhalation device as in claim 1 in which the inflatable mask is comprised of a very thin plastic bladder that is highly pliable.

9. A pediatric inhalation device comprising:
a auxiliary device including an inlet, an outlet, and a chamber;
an administration device attached to the outlet;
a coaching whistle which creates an audible sound during inhalation by a child between six months of age and four years old during effective administration of medicament from the pediatric inhalation device; and
a mask.

10. A pediatric inhalation device comprising:
a auxiliary device including an inlet, an outlet, and a chamber;
an administration device attached to the outlet;
a T-adaptor having an inhalation valve and an exhalation valve of about equal size in which the exhalation valve is operable by the flow of air produced by the normal exhalation of an infant, and the T-adaptor having a first tubular portion having a first end that attaches to the outlet of the auxiliary device and the first end contains the inhalation valve therein, and the first tubular portion having a second end which contains the exhalation valve therein, and the T-adaptor further having a second tubular portion attached perpendicularly to the first tubular portion and having an aperture at a terminal end thereof that forms a base of a mask adaptor.
a mask connected to the mask adaptor of the T-adaptor.

11. A method of encouraging and monitoring inhalation of a medicament from a pediatric inhalation device in a child between six months and three years of age including the steps of:
a. providing a whistle on the pediatric inhalation device capable of making an audible sound during desired inhalation by the child;
b. providing a mask on the pediatric inhalation device;
c. providing the pediatric inhalation device to the child for self-administration of the medicament;
d. observing the child apply the mask to his or her face to enable inhalation of the medicament; and
e. observing the child cause the audible sound from the whistle during effective inhalation of the medicament through the mask.

12. A method as in claim 11 in which said mask has highly pliable walls and is inflatable and the step of providing a mask on the pediatric inhalation device further includes the step of partially inflating the mask.

13. A method as in claim 11 further including the steps of:
a. providing a T-adaptor on the pediatric inhalation device having an inhalation valve and an exhalation valve of about equal size that are highly visible to an adult.
b. observing the valve movement of the inhalation or exhalation valve during administration of the medicament from the pediatric inhalation device.

14. A method as in claim 13 including the additional step of counting the valve movements of the inhalation valve or the exhalation valve to ensure adequate withdrawal of the medicament by the child.
